(19) United States
(12) Patent Application Publication
(10) Pub. No.: US 2020/0129492 A1
(43) Pub. Date: Apr. 30, 2020

(54) TREATMENT OF CANCER WITH DIHYDROPYRIDINES

(71) Applicant: MENRI GROUP LTD., Tel Aviv (IL)

(72) Inventor: Yehuda MATZA, Mevaseret-Zion (IL)

(21) Appl. No.: 16/627,130

(22) PCT Filed: Jul. 3, 2018
(86) PCT No.: PCT/IB2018/054927

§ 371 Date: Dec. 27, 2019

Related U.S. Application Data
(60) Provisional application No. 62/528,259, filed on Jul. 3, 2017.
(60) Provisional application No. 62/537,598, filed on Jul. 27, 2017.

(51) Int. Cl. A61K 31/4422; A61K 31/451; A61K 9/00; A61P 35/00

(52) U.S. Cl. CPC A61K 31/4422; A61P 35/00; A61K 9/0053; A61K 31/451

(57) ABSTRACT

Provided are methods and compositions for treating cancer. An embodiment of a method includes administering an effective amount of at least one dihydropyridine, such as lercanidipine, manidipine, nitrendipine, nicardipine, nisoldipine, and any combination thereof. A dihydropyridine may be administered in combination with loperamide.

### CROSS-REFERENCE TO RELATED APPLICATIONS

The present application claims priority to U.S. Provisional Patent Application Nos. 62/528,259 (filed on Jul. 3, 2017) and 62/537,598 (filed on Jul. 27, 2017), each of which is incorporated herein by reference in its entirety.

### TECHNICAL FIELD

The present disclosure relates to dihydropyridines. In particular, the present disclosure relates to using dihydropyridines to treat cancer.

### BACKGROUND

Calcium signaling is a common mechanism involved in the majority of cellular functions. Ca²⁺ homeostasis is tightly modulated by multiple channel mechanisms in all excitable cells and non-excitable cells. Calcium channels include various voltage-dependent channels, also referred to as voltage-gated channels (hereafter “Caᵥ”), and ligand-gated (receptor-operated) channels. Among the cellular functions involving Ca²⁺ signaling are many processes that mediate or regulate the development of pathologies, including cardiovascular disorders, hypertension, and types of cancer.

Caᵥ channels include several subsets that may be activated at depolarized membrane potentials. Caᵥ channels are heteromultimers composed of a pore forming α1 subunit, β regulatory subunit, α2 subunit, γ subunit, and δ subunit. The topology of the α1 pore forming subunit is predicted to have four repeated motifs (I-IV), each of which are hexahelical. The S4 transmembrane segments in each motif contain conserved positively charged amino acids that are voltage-sensors and that move outwards upon membrane depolarization, thereby opening the channel, Cav α1 subunits may be classified into three subsets having specific functions in different cell types: Caᵥ1 (L-type), Caᵥ2 (N-, P/Q- and R-type), and Caᵥ3 (T-type). There are four L-type α1 proteins: α1S (Caᵥ1.1), α1C (Caᵥ1.2), α1D (Caᵥ1.3), and α1F (Caᵥ1.4).

Caᵥ channel expression (i.e., Caᵥ1.3) has been detected in certain cancer cell lines, including common carcinomas such as prostate cancer, endometrial cancer, colon cancer, breast cancer, and lung cancer. The definitive role of these Caᵥ channels in tumor development has not been established and remains unknown.

As the role of the Caᵥ channels in cancer development is largely not understood, the reported effects of Ca²⁺ channel blockers in clinical practice have raised substantial concerns regarding their involvement in tumor development. Owing to the wide use of Ca²⁺ channel blockers in treating hypertension, multiple epidemiologic surveys were conducted to assess the risk of cancer incidence in blocker users. The results of these studies were inconsistent, and varied considerably depending on the design of the particular study. A large body of evidence implicated Ca²⁺ channel blockers with increased risk for cancer. For example, Ca²⁺ channel blockers were found to be positively associated with breast cancer risk. Other studies in prostate cancer patients reported no excessive risk of prostate cancer incidence, or even found a reverse correlation between the likelihood of prostate cancer risk and the use of Ca²⁺ channel blockers among men without family history. As the reasons and underlying mechanisms for these strikingly different effects are largely unknown, the ability to predict how a particular subject would react to a particular blocker remains a challenge. This uncertainty would preclude the use of Ca²⁺ channel blockers in cancer therapy, and indeed no such agents are currently approved for the treatment of cancer.

### SUMMARY

The present disclosure provides for a method of treating cancer or inhibiting growth of cancer cells in a subject. The method may comprise administering an effective amount of at least one dihydropyridine to the subject.

The present disclosure also provides for a method of reducing cancer burden in a subject. The method may comprise administering an effective amount of at least one dihydropyridine to the subject.

Also encompassed by the present disclosure is a method of treating cancer or inhibiting growth of cancer cells in a subject, where the subject is afflicted with a cancer expressing a Caᵥ1.3 calcium channel. The method may comprise administering an effective amount of at least one dihydropyridine to the subject.

The present disclosure provides for a method of reducing cancer burden in a subject, where the subject is afflicted with a cancer expressing a Caᵥ1.3 calcium channel. The method may comprise administering an effective amount of at least one dihydropyridine to the subject.

The method may further comprise the step of identifying the cancer in the subject. For example, the identifying step may comprise identifying whether the cancer expresses a Caᵥ1.3 calcium channel. The identifying step may comprise identifying whether the cancer expresses an isoform of a Caᵥ1.3 channel.

The cancer may be a carcinoma. In one embodiment, the cancer is a lung cancer, such as a non-small cell lung cancer.

In certain embodiments, the cancer is a tumor where the effective amount is sufficient to reduce the size or mass of the tumor.

The present disclosure provides for a method of inhibiting growth of cancer cells. The method may comprise contacting the cancer cells with an effective amount of at least one dihydropyridine.

The present disclosure also provides for a method of inhibiting growth of cancer cells, where the cancer cells express a Caᵥ1.3 calcium channel. The method may comprise contacting the cancer cells with an effective amount of at least one dihydropyridine.

In certain embodiments, the cancer cells are lung cancer cells.

The dihydropyridine may be lercanidipine, manidipine, nitrendipine, nicardipine, nisoldipine, or any combination thereof.

The dihydropyridine may be administered orally, topically, intratumorally, through a pulmonary administration, or through any other route as described herein.

In one embodiment, the effective amount of the dihydropyridine ranges from about 0.1 mg/day to about 100 mg/day.

In another embodiment, the effective amount of the dihydropyridine ranges from about 5 μM to about 50 μM.

Also encompassed by the present disclosure is a pharmaceutical composition for the treatment of cancer, the pharmaceutical composition comprising an effective amount of at least one dihydropyridine.

The dihydropyridine may be lercanidipine, manidipine, nitrendipine, nicardipine, nisoldipine, or any combination thereof.

The pharmaceutical composition may be configured to be administered orally.

The effective amount of the dihydropyridine, may range from about 0.1 to about 100 mg.

The pharmaceutical composition may be configured to be administered topically, intratumorally, or through a pulmonary administration.

The dihydropyridine may have a concentration ranging from about 5 μM to about 50 μM.

In one embodiment, the dihydropyridine is nitrendipine, where the pharmaceutical composition is configured to be administered locally, and where the dihydropyridine has a concentration ranging from about 5 μM to about 50 μM.

The present disclosure provides for a method of treating cancer comprising identifying the cancer in a subject, and administering an effective amount of at least one dihydropyridine.

The dihydropyridine may be 4-ring dihydropyridines such as lercanidipine, manidipine; 3-ring dihydropyridines, such as nicardipine, and/or 2-ring dihydropyridines such as nitrendipine and nisoldipine, and any combination thereof.

Additionally or alternatively, the method may include one or more of the following features individually or in combination: the dihydropyridine may be selected from the group consisting of lercanidipine, manidipine, nitrendipine, nicardipine, nisoldipine, and any combination thereof; the cancer may be a carcinoma; the cancer may be a lung cancer; the cancer may be a non-small cell lung cancer; the cancer may be a tumor and the effective amount may be sufficient to reduce the size or mass of the tumor; the identifying the cancer in a subject may further comprise identifying whether the cancer expresses a Caᵥ1.3 channel; the identifying the cancer in a subject may further comprise identifying whether the cancer expresses an isoform of a Caᵥ1.3 channel; a pharmaceutical composition of the dihydropyridine may be prepared prior to administering the effective amount of the dihydropyridine; the dihydropyridine may be administered orally; the effective amount of the dihydropyridine may be in a range of about 0.1 to about 100 mg/day; the dihydropyridine may be administered topically, intratumorally, or through a pulmonary administration; the effective amount of the dihydropyridine may be in a range of about 5 μM to about 50 μM.

The embodiments also relate to a pharmaceutical composition for the treatment of cancer comprising at least one dihydropyridine prepared for treatment through an administration method selected from the group consisting of topical administration, intratumoral administration, pulmonary administration, and any combination thereof.

Additionally or alternatively, the pharmaceutical composition may include one or more of the following features individually or in combination: the dihydropyridine may be selected from the group consisting of lercanidipine, manidipine, nitrendipine, nicardipine, nisoldipine, and any combination thereof; the pharmaceutical composition may be configured to be administered orally; the concentration of the dihydropyridine may be in range of about 0.1 to about 100 mg; the pharmaceutical composition may be configured to be administered topically, intratumorally, or through a pulmonary administration; the concentration of the dihydropyridine may be in a range of about 5 μM to about 50 μM; the dihydropyridine may be nitrendipine and the pharmaceutical composition may be configured to be administered locally and have a concentration in a range of about 5 μM to about 50 μM.

### BRIEF DESCRIPTION OF THE DRAWINGS

Illustrative examples of the present disclosure are described in detail below with reference to the attached drawing figures, which are incorporated by reference herein, and wherein:

FIG. 1A illustrates a concentration of intracellular calcium in A549 lung cancer cells after treatment with nitrendipine in accordance with the embodiments described herein;

FIG. 1B illustrates an effect on A549 lung cancer cell proliferation after treatment with nitrendipine in accordance with the embodiments described herein;

FIG. 2A illustrates a concentration of intracellular calcium in A549 lung cancer cells after treatment with nicardipine in accordance with the embodiments described herein;

FIG. 2B illustrates an effect on A549 lung cancer cell proliferation after treatment with nicardipine in accordance with the embodiments described herein;

FIG. 3 illustrates a concentration of intracellular calcium in A549 lung cancer cells after sequential treatment with nitrendipine and nicardipine in accordance with the embodiments described herein;

FIG. 4A illustrates a concentration of intracellular calcium in A549 lung cancer cells after treatment with nisoldipine in accordance with the embodiments described herein;

FIG. 4B illustrates an effect on A549 lung cancer cell proliferation after treatment with nisoldipine in accordance with the embodiments described herein;

FIG. 5 illustrates a concentration of intracellular calcium in A549 lung cancer cells after treatment with the drug of Formula IV in accordance with the embodiments described herein; and

FIG. 6 illustrates a comparison of A549 lung cancer cell proliferation after treatment with a nitrendipine or the drug of Formula IV in accordance with the embodiments described herein.

FIG. 7A illustrates the concentration of intracellular calcium in A549 lung cancer cells after treatment with loperamide in accordance with the embodiments described herein;

FIG. 7B illustrates the effect on A549 lung cancer cell proliferation after treatment with loperamide in accordance with the embodiments described herein;

FIG. 8 illustrates the effect on A549 lung cancer cell proliferation after treatment with a combination of loperamide and nitrendipine in accordance with the embodiments described herein; and

FIG. 9 illustrates the effect on A549 lung cancer cell proliferation after treatment with a combination of loperamide and nicardipine in accordance with the embodiments described herein.

FIG. 10 illustrates the effect on A549 lung cancer cell proliferation after treatment with nicardipine (“Nic”) or lercanidipine (“Ler”) alone, or treatment with a combination of loperamide (5 uM, “LP”) with various doses of nicardipine (“Nic”) or lercanidipine (“Ler”).

The illustrated figures are exemplary only and are not intended to assert or imply any limitation with regard to the environment, structure, form, design, or process in which different examples may be implemented.

### DETAILED DESCRIPTION

The present disclosure relates to dihydropyridines. In particular, the present disclosure relates to using dihydropyridines to treat cancer. The present disclosure relates to the preparation of pharmaceutical compositions comprising said dihydropyridines, as well as the administration of said dihydropyridines as a therapy for the treatment of cancer.

The dihydropyridine may be formulated into a pharmaceutical composition, where the dihydropyridine is present in amounts ranging from about 0.01% (w/w) to about 100% (w/w), from about 0.1% (w/w) to about 80% (w/w), from about 1% (w/w) to about 70% (w/w), from about 10% (w/w) to about 60% (w/w), or from about 0.1% (w/w) to about 20% (w/w). The present compositions can be administered alone, or may be co-administered together with radiation or another agent (e.g., a chemotherapeutic agent), to treat a disease such as cancer. Treatments may be sequential, with the dihydropyridine being administered before or after the administration of other agents. For example, a dihydropyridine may be used to sensitize a cancer patient to radiation or chemotherapy. Alternatively, agents may be administered concurrently. The route of administration may vary, and can include, inhalation, intranasal, oral, transdermal, intravenous, subcutaneous or intramuscular injection. The present disclosure also provides for a method of treating a disease such as cancer, comprising the step of delivering to a patient a therapeutically effective amount of a dihydropyridine.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification, including the attached claims are approximations that may vary depending upon the desired properties sought to he obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit. The term “about” in reference to a numeric value refers to ±10% of the stated numeric value. In other words, the numeric value can be in a range of 90% of the stated value to 110% of the stated value.

In some embodiments, a dihydropyridine may be provided. In further embodiments, the present composition may contain one or more types of dihydropyridine. In further embodiments, an effective amount of the dihydropyridine may be administered to a human to treat cancer.

A dihydropyridine is a pyridine derivative and a starting material for a class of molecules that have been semi-saturated with two substituents replacing a double bond. Some examples are known in pharmacology as L-type calcium channel blockers, and have been used in the treatment of hypertension. Compared with certain other L-type calcium channel blockers (e.g., phenylalkylamines), dihydropyridines are relatively vascular selective in their mechanism of action in lowering blood pressure. Examples of dihydropyridines may include, but are not limited to, amlodipine, aranidipine, barnidipine, henidipine, cilnidipine, clevidipine, cronidipine, darodipine, dexniguldipine, efonidipine, elgodipine, elnadipine, felodipine, flordipine, furnidipine, iganidipine, lacidipine, lemildipine, lercanidipine, levamlodipine, levniguldipine, manidipine, nicardipine, nifedipine, niguldipine, niludipine, nimodipine, nisoldipine, nitrendipine, olradipine, oxodipine, palonidipine, pranidipine, ryodipine, sagandipine, sornidipine, teludipine, tiamdipine, trombodipine, vatanidipine, isomers thereof, derivatives thereof, or any combination thereof.

In some exemplary embodiments, nitrendipine is provided as the dihydropyridine. The skeletal structure of nitrendipine (IUPAC name: (RS)-3-Ethyl 5-methyl 2,6-dimethyl-4-(m-nitrophenyl)-1,4-dihydropyridine-3,5-dicarboxylate) is illustrated in Formula I below:

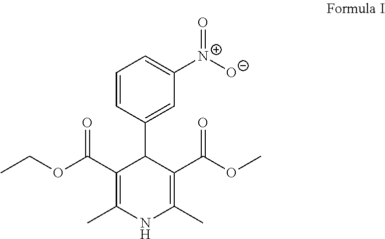 [SMILES: CCOC(=O)C1=C(C)NC(C)=C(C(=O)OC)C1c1cccc([N+](=O)[O-])c1]

Nitrendipine may be used for the treatment of hypertension. As noted above, nitrendipine is a dihydropyridine calcium channel blocker. After ingestion, nitrendipine is absorbed by the gut and metabolized by the liver before it goes into the systemic circulation and reaches the cells of the smooth muscles and cardiac muscle cells. In hypertension, the binding of nitrendipine causes a decrease in the open probability of L-type calcium channels and reduces the influx of calcium. The reduced levels of calcium prevent smooth muscle contraction within these muscle cells. Prevention of muscle contraction enables smooth muscle dilation. Dilation of the vasculature reduces total peripheral resistance, which decreases the workload on the heart and prevents scarring of the heart or heart failure.

In some exemplary embodiments, nicardipine is provided as the dihydropyridine. The skeletal structure of nicardipine (IUPAC name: 2-[benzyl(methyl)amino]ethylmethyl-2,6-dimethyl-4-(3-nitrophenyl)-1,4-dihydropyridine-3,5-dicarboxylate) is illustrated in Formula II below:

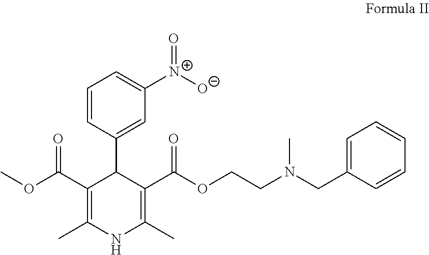 [SMILES: COC(=O)C1=C(C)NC(C)=C(C(=O)OCCN(C)Cc2ccccc2)C1c1cccc([N+](=O)[O-])c1]

Nicardipine may be used for the treatment of angina, hypertension and Raynaud's phenomenon. As noted above, nicardipine is a dihydropyridine calcium channel blocker. After ingestion, nicardipine is absorbed by the gut and metabolized by the liver before it goes into the systemic circulation and reaches the cells of the smooth muscles and cardiac muscle cells. Nicardipine inhibits the movement of calcium ions into smooth muscle cells and cardiac muscle cells. The contractile processes of cardiac muscle and smooth muscle are dependent upon the movement of extracellular calcium ions into these cells through specific Caᵥ channels. Calcium channel blockers interfere with this movement. The effect of this interference is to relax blood vessels, widening them in turn. This may lower blood pressure and reduce stress on the heart.

In some exemplary embodiments, nisoldipine is provided as the dihydropyridine. The skeletal structure of nisoldipine (IUPAC name: isobutyl methyl 2,6-dimethyl4-(2-nitrophenyl)-1,4-dihydropyridine-3,5-dicarboxylate) is illustrated in Formula III below:

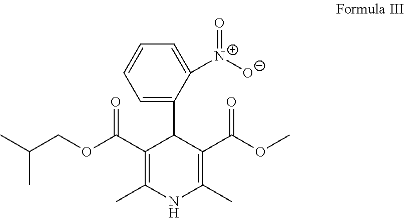 [SMILES: COC(=O)C1=C(C)NC(C)=C(C(=O)OCC(C)C)C1c1ccccc1[N+](=O)[O-]]

Nisoldipine's most common use is for the treatment of hypertension. As noted above, nitrendipine is a dihydropyridine calcium channel blocker. As with similar dihydropyridine calcium channel blockers, nisoldipine is absorbed by the gut after ingestion and then metabolized by the liver. Afterwards, the metabolic product is then delivered to the systemic circulation where it may reach the smooth muscle cells and the cardiac muscle cells. In hypertension, the binding of nisoldipine causes a decrease in the probability of open L-type calcium channels and reduces the influx of calcium. The reduced levels of calcium prevent smooth muscle contraction within these muscle cells. Prevention of muscle contraction enables smooth muscle dilation. Dilation of the vasculature reduces total peripheral resistance, which decreases the workload on the heart and prevents scarring of the heart or heart failure.

In some exemplary embodiments, the dihydropyridine is 5-O-ethyl 3-O-methyl 2,6-dimethyl-4-phenyl-1,4-dihydropyridine-3,5-dicarboxylate. The skeletal structure of 5-O-ethyl 3-O-methyl 2,6-dimethyl-4-phenyl-1,4-dihydropyridine-3,5-dicarboxylate is illustrated in Formula IV below:

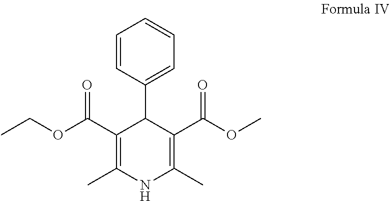 [SMILES: CCOC(=O)C1=C(C)NC(C)=C(C(=O)OC)C1c1ccccc1]

Compared to nitrendipine, 5-O-ethyl 3-O-methyl 2,6-dimethyl-4-phenyl-1,4-dihydropyridine-3,5-dicarboxylate possesses a phenyl group in place of a nitrophenyl group and this substitution may alter the effect of the 5-O-ethyl 3-O-methyl 2,6-dimethyl-4-phenyl-1,4-dihydropyridine-3,5-dicarboxylate on some Caᵥ channels, both with regards to the ability of the 5-O-ethyl 3-O-methyl 2,6-dimethyl-4-phenyl-1,4-dihydropyridine-3,5-dicarboxylate to bind the Caᵥ channel and with the conformational change exhibited by the Caᵥ channel when the 5-O-ethyl 3-O-methyl 2,6-dimethyl-4-phenyl-1,4-dihydropyridine-3,5-dicarboxylate binds to a receptor site in the Caᵥ channel.

In some exemplary embodiments, lercanidipine is provided as the dihydropyridine. The skeletal structure of lercanidipine (IUPAC name: (RS)-2[(3,3-Diphenylpropyl)(methyl)amino]-1,1-dimethylethyl methyl 2,6-dimethyl-4-nitrophenyl)-1,4-dihydropyridine-3,5-dicarboxylate) is illustrated in Formula V below:

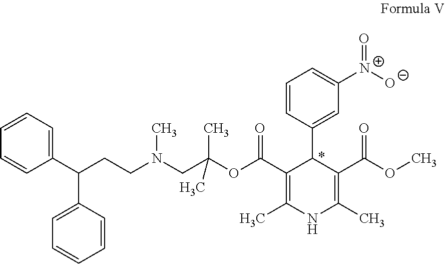 [SMILES: COC(=O)C1=C(C)NC(C)=C(C(=O)OC(C)(C)CN(C)CCC(c2ccccc2)c2ccccc2)C1c1cccc([N+](=O)[O-])c1]

An effective amount of a dihydropyridine may be used to treat some types of cancer. Without limitation by theory, it is theorized that some types of cancer cells express isoforms of the Caᵥ1.3 channel. These cancer-specific isoforms may possess a gating mechanism different than the Caᵥ1.3 channel of normal cells. As such, the interaction of traditional Caᵥ1.3 channel agonists/antagonists with the isoform Caᵥ1.3 channels may be different than expected. For example, a traditional dihydropyridine calcium channel blocker, may instead lethally flood a cancer cell comprising the isoform Caᵥ1.3 channels with calcium. Alternatively, the dihydropyridine calcium channel blocker may inhibit the isoform Caᵥ1.3 channel to such an extent that the cancer cell cannot initiate or successfully finish cytokinesis, and this may result in cell death.

Without limitation by theory, the unexpected result of cell death was observed in cancer cells after treatment with a dihydropyridine. This unexpected result was especially pronounced at lower concentrations of the dihydropyridine as discussed below. Further, these unexpected results may be due to the interaction of the dihydropyridine with isoforms of the Caᵥ channels (e.g., isoforms of the Caᵥ1.3 channels) that are expressed in cancer cells. In some embodiments, these isoforms may not be expressed in the corresponding non-cancerous cells. The isoforms may be formed from any method of alternative splicing. Without limitation by theory, and as noted above, the structural differences in the isoform voltage channels may allow the dihydropyridine to interact with the subject cell in a different manner than it does with the traditional voltage channel. As such, a calcium channel blocker as described herein (such as nitrendipine) may instead open the isoform voltage channel and lethally flood the cell with extracellular calcium. Alternatively, a calcium channel blocker as described herein (such as nisoldipine) may block the isoform voltage channel to such a degree that the cell is deprived of sufficient calcium and cannot initiate or successfully finish cytokinesis resulting in cell death. These results are unexpected as typical dihydropyridines are well tolerated and not known to be especially cytotoxic at the concentrations prescribed for other maladies. Further, these results are unexpected as the observed cytotoxicity in the cancer cell line may result from treatment with the dihydropyridines alone and without any other drugs or chemotherapeutic agents. As such, some embodiments comprise the administration of a pharmaceutical composition comprising a dihydropyridine, or salt or derivative thereof, for the treatment of cancer. The treatment may be administration of a pharmaceutical composition comprising a dihydropyridine (or salt or derivative thereof) alone without the administration of other drugs or chemotherapeutic agents. The treatment may be administration of a pharmaceutical composition comprising a dihydropyridine (or salt or derivative thereof) in combination with other treatment(s)/agent(s).

The present composition may be administered alone or in combination with a second agent/treatment method (therapeutic intervention).

In certain embodiments, the present method for treating cancer may comprise the step of administering to a subject a dihydropyridine (or salt or derivative thereof) in combination with other treatment(s)/agent(s) such as an opioid or opioid-receptor agonist (e.g., loperamide). In certain embodiments, the present method for treating cancer cells may comprise the step of administering to the cancer cells a dihydropyridine (or salt or derivative thereof) in combination with other treatment(s)/agent(s) such as an opioid or opioid-receptor agonist (e.g., loperamide).

The skeletal structure of loperamide (IUPAC name: 4-[4-(4-Chlorophenyl)-4-hydroxypiperidin-1-yl]-N,N-dimethyl-2,2-diphenylbutanamide) is illustrated in Formula VI below:

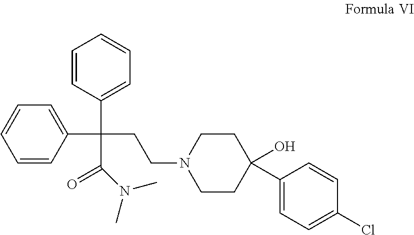 [SMILES: CN(C)C(=O)C(CCN1CCC(O)(c2ccc(Cl)cc2)CC1)(c1ccccc1)c1ccccc1]

Loperamide's most common use is for the treatment of diarrhea. As a diarrhea medication, loperamide functions as an opioid-receptor agonist that acts on the μ-opioid receptors in the myenteric plexus of the large intestine. Loperamide decreases the activity of the myenteric plexus, which decreases the tone of the longitudinal and circular smooth muscles of the intestinal wall. This in turn increases the time any material may reside in the intestine, allowing more water to be absorbed from the fecal matter. Loperamide also decreases colonic mass movements and suppresses the gastrocolic reflex. Loperamide is extremely well tolerated even though it is an opioid because it has functionally extremely low absorption into the gut (i.e., it does not substantially circulate in the bloodstream) and does not cross the blood-brain barrier. Loperamide's circulation in the bloodstream is limited in two ways. Efflux by P-glycoprotein in the intestinal wall reduces passage of loperamide, and any fractional amount of drug crossing may be further reduced through first pass metabolism by the liver, where loperamide is metabolized into other compounds.

The combination therapy may be achieved by administering a pharmaceutical composition that includes both agents (a dihydropyridine (or salt or derivative thereof) and an opioid or opioid-receptor agonist (e.g., loperamide)), or by administering two pharmaceutical compositions, at the same time or within a short time period, wherein one composition comprises a dihydropyridine (or salt or derivative thereof), and the other composition includes an opioid or opioid-receptor agonist loperamide).

The combination of the a dihydropyridine (or salt or derivative thereof) and an opioid or opioid-receptor agonist (e.g., loperamide) may produce an additive or synergistic effect (i.e., greater than additive effect) in treating the cancer or cancer cells compared to the effect of the dihydropyridine (or salt or derivative thereof) or the an opioid or opioid-receptor agonist (e.g., loperamide) alone. For example, the combination may result in a synergistic increase in apoptosis of cancer cells, and/or a synergistic reduction in tumor volume. In different embodiments, depending on the combination and the effective amounts used, the combination of compounds can inhibit tumor growth, achieve tumor stasis, or achieve substantial or complete tumor regression.

The present disclosure provides methods to reduce cancer cell growth, proliferation, and/or metastasis, as measured according to routine techniques in the diagnostic art. Specific examples of relevant responses include reduced size, mass, or volume of a tumor, or reduction in cancer cell number.

The present compositions and methods can have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; preventing, inhibiting or ameliorating the cancer in the subject, such as slowing progression of the cancer, reducing or ameliorating a sign or symptom of the cancer; reducing the rate of tumor growth in a patient; preventing the continued growth of a tumor, reducing the size of a tumor; and/or disappearance of markers associated with undesirable, unwanted, or aberrant cell proliferation. U.S. Patent Publication No. 20080275057 (incorporated herein by reference in its entirety).

Methods and compositions of the present invention can be used for prophylaxis as well as amelioration of signs and/or symptoms of cancer.

In some embodiments, the combination therapy results in a synergistic effect, for example, the dihydropyridine (or salt or derivative thereof) and an opioid or opioid-receptor agonist (e.g., loperamide) act synergistically, for example, in the apoptosis of cancer cells, inhibition of proliferation/survival of cancer cells, in the production of tumor stasis.

As used herein, the term “synergy” (or “synergistic”) means that the effect achieved with the methods and combinations of this disclosure is greater than the sum of the effects that result from using the individual agents alone, e.g., using the dihydropyridine (or salt or derivative thereof) alone and the opioid or opioid-receptor agonist (e.g., loperamide) alone. For example, the effect (e.g., apoptosis of cells, a decrease in cell viability, cytotoxicity, a decrease in cell proliferation, a decrease in cell survival, inhibition of tumor growth, a reduction in tumor volume, and/or tumor stasis, etc. as described herein) achieved with the combination of a dihydropyridine (or salt or derivative thereof) and an opioid or opioid-receptor agonist (e.g., loperamide) is about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 12 fold, about 15 fold, about 20 fold, about 25 fold, about 30 fold, about 50 fold, about 100 fold, at least about 1.2 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 8 fold, at least about 9 fold, at least about 10 fold, of the sum of the effects that result from using the dihydropyridine (or salt or derivative thereof) alone or the opioid or opioid-receptor agonist (e.g., loperamide) alone.

Synergistic effects of the combination may also be evidenced by additional, novel effects that do not occur when either agent is administered alone, or by reduction of adverse side effects when either agent is administered alone.

Cytotoxicity effects can be determined by any suitable assay, including, but not limited to, assessing cell membrane integrity (using, e.g., dyes such as trypan blue or propidium iodide, or using lactate dehydrogenase (LDH) assay), measuring enzyme activity, measuring cell adherence, measuring ATP production, measuring co-enzyme production, measuring nucleotide uptake activity, crystal violet method, Tritium-labeled Thymidine uptake method, measuring lactate dehydrogenase (LDH) activity, 3-(4,5-Dimethyl-2-thiazolyl)-2,5-diphenyl-2H-tetrazolium bromide (MTT) or MTS assay, sulforhodamine B (SRB) assay, WST assay, clonogenic assay, cell number count, monitoring cell growth, etc.

Apoptosis of cells may be assayed by any suitable method, including, but not limited to, TUNEL (terminal deoxynucleotidyl transferase dUTP nick end labeling) assay, assaying levels of cytochrome C release, assaying levels of cleaved/activated caspases, assaying 5-bromo-2′-deoxyuridine labeled fragmented DNA, assaying levels of survivin etc.

Other methods that can be used to show the synergistic effects of the present methods, pharmaceutical compositions and combinations include, but are not limited to, clonogenic assay (colony formation assay) to show decrease in cell survival and/or proliferation, studying tumor volume reduction in animal models (such as in mice, etc.)

In one embodiment, advantageously, such synergy provides greater efficacy at the same doses, lower side effects, and/or prevents or delays the build-up of multi-drug resistance.

The dihydropyridine (or salt or derivative thereof) and an opioid or opioid-receptor agonist loperamide) may be administered simultaneously, separately or sequentially. They may exert an advantageously combined effect (e.g., additive or synergistic effects).

For sequential administration, either a dihydropyridine (or salt or derivative thereof) is administered first and then an opioid or opioid-receptor agonist (e.g., loperamide), or the opioid or opioid-receptor agonist (e.g., loperamide) is administered first and then a dihydropyridine (or salt or derivative thereof). In embodiments where a dihydropyridine (or salt or derivative thereof) and an opioid or opioid-receptor agonist (e.g., loperamide) are administered separately, administration of a first agent can precede administration of a second agent by seconds, minutes, hours, days, or weeks. The time difference in non-simultaneous administrations may be greater than 1 minute, and can be, for example, precisely, at least, up to, or less than 5 minutes, 10 minutes, 15 minutes, 30 minutes, 45 minutes, 60 minutes, 2 hours, 3 hours, 6 hours, 9 hours, 12 hours, 24 hours, 36 hours, or 48 hours, or more than 48 hours. The two or more agents can be administered within minutes of each other or within about 0.5, about 1, about 2, about 3, about 4, about 6, about 9, about 12, about 15, about 18, about 24, or about 36 hours of each other or within about 1, 2, 3, 4, 5, 6, 7, 8, 9, 10, 12, 14 days of each other or within about 2, 3, 4, 5, 6, 7, 8, 9, or 10 weeks of each other. In some eases, longer intervals are possible.

The present disclosure also provides for a pharmaceutical composition comprising (i) a dihydropyridine (or salt or derivative thereof); and (ii) an opioid or opioid-receptor agonist (e.g., loperamide).

The present compositions may be administered alone, or in combination with radiation, surgery or chemotherapeutic agents. The present compositions may be administered before, during or after the administration of radiation, surgery or chemotherapeutic agents.

The present disclosure also provides for methods of using a dihydropyridine to treat a disease, such as cancer. A dihydropyridine may be administered alone, or in combination with radiation, surgery or chemotherapeutic agents. A dihydropyridine may also be co-administered with antiviral agents, anti-inflammatory agents or antibiotics. The agents may be administered concurrently or sequentially. A dihydropyridine can be administered before, during or after the administration of the other active agent(s).

The dihydropyridine may be used in combination with radiation therapy. In one embodiment, the present disclosure provides for a method of treating tumor cells or cancer with radiation, where the cells are treated with an effective amount of a dihydropyridine, and then exposed to radiation. Dihydropyridine treatment may be before, during and/or after radiation. For example, the dihydropyridine may be administered continuously beginning one week prior to the initiation of radiotherapy and continued for two weeks after the completion of radiotherapy.

In one embodiment, the present invention provides for a method of treating tumor cells or cancer with chemotherapy, where the cells are treated with an effective amount of a dihydropyridine, and then exposed to chemotherapy. Dihydropyridine treatment may be before, during and/or after chemotherapy.

The present agent/composition may be used in combination with a cytotoxic agent. The combination of the present agent/composition and the cytotoxic agent may produce a synergistic effect on the cancer or cancer cells compared to the effect of the present agent/composition alone or the effect of the cytotoxic agent atone. The synergist effects are discussed herein.

The cytotoxic agent may be any chemotherapeutic agents including, but not limited to, alkylating agents, anti-metabolites, anti-microtubule agents, topoisomerase, inhibitors, cytotoxic antibiotics, endoplasmic reticulum stress inducing agents, platinum compounds, vincalkaloids, taxanes, epothilones, enzyme inhibitors, receptor antagonists, tyrosine kinase inhibitors, boron radiosensitizers (i.e. velcade), and chemotherapeutic combination therapies.

Non-limiting examples of DNA alkylating agents are nitrogen mustards, such as Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, Triaziquone, Triethylenemelamine; Hydrazines (Procarbazine); Triazenes such as Dacarbazine and Temozolomide (TMZ); Altretamine and Mitobronitol.

Non-limiting examples of Topoisomerase I inhibitors include Campothecin derivatives including SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6(10):789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):31913194, and Crespi et al. (1986) Biochem. Biophys. Res. Commun. 136(2):521-8, Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other Benzopyridoindoles, TAS-103 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]pyrimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Platinum based compounds are a subclass of DNA alkylating agents. Non-limiting examples of such agents include Cisplatin, Nedaplatin, Oxaliplatin, Triplatin tetranitrate, Satraplatin, Aroplatin, Lobaplatin, and JM-216. (See McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Non-limiting examples of antimetabolite agents include folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Hudarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-I (MBMS-247616, consisting of tegafur and two modulators, a 5-chloro-2,4-dihydroxypyridine and potassium oxonate), ralititrexed (tomudex), nolatrexed (Thymitaq, A0337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Paclitaxel and Tesetaxel. An example of an epothilone is iabepilone.

Examples of enzyme inhibitors include, but are not limited to farnesyltransferase inhibitors (e.g., Tipifarnib); CDK inhibitors (e.g., Alvocidib, Seliciclib); proteasome inhibitors (e.g., Bortezomib); phosphodiesterase inhibitors (e.g., Anagrelide; rolipram); IMP dehydrogenase inhibitors (e.g., Tiazofurine); and lipoxygenase inhibitors (e.g., Masoprocol).

Chemotherapeutic agents may also include amsacrine, Trabectedin, retinoids (Alitretinoin, Tretinoin), Arsenic trioxide, asparagine depicter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

Cancers treated using methods and compositions described herein are characterized by abnormal cell proliferation including, but not limited to, pre-neoplastic hyperproliferation, cancer in-situ, neoplasms and metastasis. The term “cancer” as used herein means any proliferative disorder wherein the cells abnormally divide and optionally invade or spread to nearby and/or distant tissues. The phrase “cancer burden” refers to the quantum of cancer cells or cancer volume in a subject. Reducing cancer burden accordingly refers to reducing the number of cancer cells or the cancer volume in a subject.

Cancers that can be treated by the present compositions and methods include, but are not limited to, lung cancer (e.g., non-small cell lung cancer), melanoma, breast cancer, colorectal cancer, pancreatic cancer, cervical cancer, thyroid cancer, bladder cancer, liver cancer, prostate cancer, muscle cancer, hematological malignancies, endometrial cancer, lymphomas, sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, synovioma, mesothelioma, lymphangioendotheliosarcoma, Ewing's tumor, leiomyosarcoma, rhabdomyosarcoma, colon carcinoma, ovarian cancer, gastric cancer, esophageal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e,g., acute lymphocytic leukemia and acute myelocytic, leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, ear, nose and throat cancer, hematopoietic cancer, biliary tract cancer; bladder cancer; bone cancer; choriocarcinoma; connective tissue cancer; cancer of the digestive system; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; lymphoma including Hodgkin's and Non-Hodgkin's lymphoma; myeloma; fibroma, oral cavity cancer (e.g., lip, tongue, mouth, and pharynx); prostate cancer; retinoblastoma; rhabdomyosarcoma; rectal cancer; renal cancer; cancer of the respiratory system; skin cancer; stomach cancer; testicular cancer; uterine cancer; cancer of the urinary system, as well as other carcinomas and sarcomas.

The present disclosure provides a pharmaceutical composition (or pharmaceutical formulation) comprising a dihydropyridine, or a pharmaceutically acceptable salt thereof, and one or more excipients (also referred to as carriers and/or diluents in the pharmaceutical arts).

Also encompassed by the present disclosure is a pharmaceutical composition comprising a dihydropyridine (or salt or derivative thereof) and an opioid or opioid-receptor agonist (e.g., loperamide).

In alternative embodiments, two or more individual pharmaceutical compositions may be prepared. The individual pharmaceutical compositions comprise loperamide or the dihydropyridine, or pharmaceutically acceptable salts thereof, and one or more excipients. The two or more individual pharmaceutical compositions may then be delivered to the desired subject (e.g., a human, tissue, etc.) or cells in combination.

In one embodiment, the synergistic effect is especially pronounced at lower concentrations of a dihydropyridine (or salt or derivative thereof) and/or an opioid or opioid-receptor agonist loperamide).

The excipients are acceptable in the sense of being compatible with the other ingredients of the formulation and not deleterious to the recipient thereof (i.e., the patient). Further embodiments provide the administration of an effective amount or a therapeutically effective amount of the dihydropyridine or a pharmaceutical composition of the dihydropyridine.

The purpose of a pharmaceutical composition is to facilitate administration of the dihydropyridine to a subject (e.g., a human). The pharmaceutical compositions may be formulated by one having ordinary skill in the art. Suitable pharmaceutical carriers include, but are not limited to, fillers, disintegrants, lubricants, glidants, and soluble and insoluble polymers, examples of which are described in Remington's Pharmaceutical Sciences, A. Osol, a standard reference text in this field, which is incorporated herein in its entirety by reference. The pharmaceutical compositions of the invention are suitable for administration systemically or in a local manner, for example, via injection of the pharmaceutical composition directly into a tissue region of a patient (e.g., intralesional injection).

The present agent may refer to a dihydropyridine (or salt or derivative thereof), a combination of a dihydropyridine (or salt or derivative thereof) and an opioid or opioid-receptor agonist (e.g., loperamide), or a combination of any agents described herein. The present composition may refer to a pharmaceutical composition comprising the present agent.

Some embodiments further provide a process for the preparation of a pharmaceutical composition comprising combining, reacting, mixing (or admixing), etc. the dihydropyridine, or salt thereof, with at least one excipient.

Typically, but not absolutely, the salts of the present invention are pharmaceutically acceptable salts. Salts encompassed within the term “pharmaceutically acceptable salts” refer to non-toxic salts of a dihydropyridine. Salts of the dihydropyridine may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts may include acetate, henzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which may not be pharmaceutically acceptable, may still be useful in the preparation of pharmaceutical compositions of the dihydropyridine, and these should be considered to form a further aspect of the embodiments. These salts, such as oxalic or trifluoroacetate, while not in themselves pharmaceutically acceptable, may be useful in the preparation of salts used as intermediates in obtaining a dihydropyridine and/or its pharmaceutically acceptable salt.

Pharmaceutical compositions may be in unit dose form containing a predetermined amount of active ingredient per unit dose. Such a unit may contain a therapeutically effective amount of a dihydropyridine. In some embodiments, a fraction of a therapeutically effective dose may be used, such that multiple unit dosage forms might be administered at a given time to achieve the desired therapeutically effective dose. Furthermore, such pharmaceutical compositions may be prepared by any of the methods well known in the pharmacy art.

Pharmaceutical compositions may be adapted for administration by any appropriate route, including for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

For injection, the active ingredients of the pharmaceutical composition may be formulated in aqueous solutions, preferably in physiologically compatible buffers such as Hank's solution, Ringer's solution, or physiological salt buffer. Pharmaceutical compositions for parenteral administration include aqueous solutions of the active preparation in water-soluble form. Additionally, suspensions of the active ingredients may be prepared as oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions. Alternatively, the active ingredient may be in powder form for constitution with a suitable vehicle, such as, for example, a sterile, pyrogen-free, water-based solution, before use.

For oral administration, the pharmaceutical composition may be formulated readily by combining the dihydropyridine with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use may be made using a solid excipient, optionally grinding the resulting mixture, and processing the mixture of granules, after adding suitable auxiliaries as desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone, hereafter “PVP.” If desired, disintegrating agents, such as cross-linked polyvinyl pyrrolidone, agar, or alginic acid or a salt thereof, such as sodium alginate, may be added.

Pharmaceutical compositions that may be used orally include push-fit capsules made of gelatin, as well as soft, sealed capsules made of gelatin and a plasticizer, such as glycerol or sorbitol. The push-fit capsules may contain the dihydropyridine in admixture with a filler such as lactose, binders such as starches, lubricants such as talc or magnesium stearate, and, optionally, stabilizers. In soft capsules, the dihydropyridine may be dissolved or suspended in suitable liquids, such as fatty oils, liquid paraffin, or liquid polyethylene glycols. In addition, stabilizers may be added in some optional embodiments. All formulations for oral administration should be in dosages suitable for the chosen route of administration.

The pharmaceutical compositions of the invention are also useful for topical and intralesional application. As used herein, the term “topical” means pertaining to a particular surface area, and the topical agent applied to a certain area of said surface will affect only the area to which it is applied.

Topical pharmaceutical compositions may comprise, without limitation, non-washable (water-in-oil) creams or washable (oil-in-water) creams, ointments, lotions, gels, suspensions, aqueous or cosolvent solutions, salves, emulsions, coated bandages or other polymer coverings, sprays, aerosols, liposomes and any other pharmaceutically acceptable carrier suitable for administration of the dihydropyridine topically.

As is well known in the art, the physico-chemical characteristics of the carrier may be manipulated by addition of a variety of excipients, including, but not limited to, thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected should preferably enhance, and in any case must not interfere with, the storage stability of the formulations.

In some embodiments, the pharmaceutical composition may be formulated for pulmonary administration. In another embodiment, the pharmaceutical composition of the dihydropyridine is formulated for administration as an aerosol or mist. In another embodiment, said pharmaceutical composition is formulated for use with a nebulizer or inhaler. The dihydropyridine may be administered by inhalation in different ways, such as in pressurized metered-dosage inhalers, in dry powder inhalers, in a liquid solution delivered by nebulizer or small volume liquid inhaler, or in a vaporized formulation suitable for inhalation or nasal aspiration. Pressurized metered dose inhalers (hereafter “pMDIs”) containing the dihydropyridine, alone or in combination with other therapeutics, with propellants, for example, may be formulated to contain the dihydropyridine in solution or in dispersion in a propellant, such as HFA 134a or HFA227, alone or in combination with excipients to modify aerosol performance, such as co-solvents (e.g., ethanol, glycerol, polyethylene glycols, propylene glycol), surfactants (e.g., oleic acid) or other excipients such as stabilizers and pH modifiers (e.g., ascorbic acid, sodium edetate, hydrochloric acid). Where the dihydropyridine is presented as a dispersion in pMDIs, then appropriate physical and/or chemical methods may be used to ensure that the aerodynamic particle size upon aerosolization is appropriate for delivery to the respiratory airways, typically less than 10 μm and preferably less than 5 μm.

Dry powder inhalers (hereafter “DPIs”) containing the dihydropyridine, alone or in combination with other therapeutics, may be formulated to contain dihydropyridine as small particles, either alone or in combination with a carrier particle such as lactose or sucrose, to aid aerosolization. Appropriate physical and/or chemical methods may be used to ensure that the aerodynamic particle size upon aerosolization from DPIs is appropriate for delivery to the respiratory airways, typically less than 10 μm and preferably less than 5 μm.

Nebulizers and small volume liquid inhaler preparations of the dihydropyridine, alone or in combination with other therapeutics, for example, may be formulated to contain the dihydropyridine in solution or in dispersion in an aqueous medium, alone or in combination with excipients to modify aerosol performance, such as co-solvents (e.g., ethanol, glycerol, polyethylene glycols, propylene glycol), surfactants (e.g., oleic acid), or other excipients such as stabilizers and modifiers (e.g., ascorbic acid, sodium edetate, hydrochloric acid). Where the dihydropyridine is presented as a dispersion in nebulizers and small volume liquid inhalers, then appropriate physical and/or chemical methods may be used to ensure that the aerodynamic particle size upon aerosolization is appropriate for delivery to the respiratory airways, typically less than 10 μm and preferably less than 5 μm.

Vaporized formulations of the dihydropyridine, alone or in combination with other therapeutics, suitable for inhalation, for example, may be formulated by heating the dihydropyridine to a high temperature for a short time period, typically less than 1 second, alone or in combination with excipients to modify aerosol performance (e.g., propylene glycol, ethanol). The methods used may ensure that the aerodynamic particle size upon aerosolization is appropriate for delivery to the respiratory airways, typically less than 10 μm and preferably less than 5 μm.

It is to be understood that any reference to the preparation of or use of any of the dihydropyridines necessarily also encompasses any preparation of or use of the salts of any of the dihydropyridines as well as the pharmaceutical compositions of any of the dihydropyridines, whether prepared alone or in combination with other dihydropyridines or other therapeutics.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a dihydropyridine, as well as any salts thereof, may be administered as the raw chemicals. Additionally, the dihydropyridine may be presented as a pharmaceutical composition.

In some embodiments, a method of treatment is provided for a human suffering from disease conditions targeted by the dihydropyridine. Such treatment comprises the step of administering to a subject an effective amount and/or therapeutically effective amount of the dihydropyridine.

In one specific embodiment, a method is provided for treating a subject (e.g., a human) having a cancer expressing an isoform of the Caᵥ1.3 channel. Said method comprises administering to the subject an effective amount and/or therapeutically effective amount of the present agent/composition, as described herein. In a further embodiment, said cancer comprising the isoform of the Caᵥ1.3 channel is identified prior to administration of the present agent/composition. For example, the method may comprise identifying a subject amenable for treatment with the present agent/composition by determining whether a biological sample of the subject expresses an isoform of the Caᵥ1.3 channel amenable for treatment with the present agent/composition. In further embodiments, the present agent/composition may then be administered locally to the tissue, organ, etc. of the subject expressing the Caᵥ1.3 isoform channel or may be administered systemically to the subject.

In various embodiments of the diagnostic methods disclosed herein, the sample may include, without limitation, a cell sample, a tissue sample, or a fluid sample. In a particular embodiment, the sample is a tumor sample (e.g., a tumor biopsy). In other embodiments, a variety of immunoassays may be used (e.g., enzyme-linked immunosorbent assay, ELISA) and/or other molecular biology assays (e.g., reverse transcription polymerase chain reaction, RT-PCR). In another embodiment, there is provided a kit for determining whether a subject is amenable for treatment with the dihydropyridine. For example, without limitation, the kit may comprise one or more antibodies, PCR primers, or other reagents that may be employed in various immunoassays and other molecular biology assays known in the art.

The present disclosure provides for a system for screening of candidate drugs for therapy with the present agent/composition. The effect of the candidate drug on a biological activity of one or more cells may be determined. In some embodiments, the biological activity is cell proliferation. In other embodiments, the biological activity is calcium influx. For example, the system may comprise a means for determining whether the test substance decreases proliferation of the one or more cells, including, but not limited to, reagents and agents for determining DNA synthesis, metabolic markers, proliferation markers, or ATP levels.

As noted above, in some embodiments, the cancer to be treated comprises a tumor. In further embodiments, the tumor is a solid tumor (e.g., a lung tumor or a breast tumor). In some examples, the tumor may be a solid tumor derived from non-excitable cells, including, but not limited to, tumors of epithelial or fibroblast origin. In another embodiment, the tumor may be derived from excitable cells such as neurons. In another embodiment, said tumor is a carcinoma. In a particular embodiment, said tumor is a lung carcinoma. In a further particular embodiment, said tumor is a non-small cell lung cancer tumor, hereafter “NSCLC.” In another embodiment, said tumor is a breast ductal carcinoma. According to some other embodiments, the methods described herein may be used for treating an established tumor in said subject with an effective amount and/or therapeutically effective amount (e.g., by reducing tumor size and/or volume), wherein each possibility represents a separate embodiment. Thus, in another embodiment, treating said subject comprises reducing tumor size and/or volume in said subject.

In some, embodiments, the present agent/composition may be administered orally. In another embodiment, the present agent/composition is administered by topical, intratumoral, of pulmonary administration. In a specific embodiment, the present agent/composition is administered topically. In another embodiment, the tumor is derived from a tissue or organ lacking Caᵥ1.3 expression. In yet another embodiment, the tumor is derived from a tissue or organ expressing neuronal type Caᵥ1.3. In another embodiment, the tumor is derived from a tissue or organ expressing Caᵥ1.3. In a still further embodiment, the tumor expresses an isoform of Caᵥ1.3.

In a specific embodiment, the present agent/composition is formulated for pulmonary administration. In a further embodiment, the present agent/composition is formulated for administration as an aerosol or mist. In a still further embodiment, said present agent/composition is formulated for use with a nebulizer or inhaler.

The precise effective amount or therapeutically effective amount of the present agent/composition will depend on a number of factors, including, but not limited to, the age and weight of the subject (patient) being treated, the precise disorder requiring treatment and its severity, the nature of the pharmaceutical formulation/composition, the route of administration, etc. The precise effective amount or therapeutically effective amount of the present agent/composition will ultimately be at the discretion of the attending physician. Typically, the precise effective amount or therapeutically effective amount of the dihydropyridine may be administered in daily oral dosages of from about 0.1 to about 1000 mg/day, and preferably from about 0.1 to about 100 mg/day. In one embodiment, the concentration is calculated as a function of the subject weight. In said embodiment, the daily oral dosage may range from about 0.05 mg/kg/day to about 0.5 mg/kg/day. As demonstrated herein, the precise effective amount or therapeutically effective amount of the dihydropyridine was found to inhibit cell proliferation at concentrations of 5-50 μM in vitro, and to inhibit tumor development at daily doses of 2-10 mg/kg in a murine model in vivo. Accordingly, exemplary doses for pharmaceutical compositions of the invention may be about 5 μM to about 50 μM for local (e.g., topical, intratumoral/intralesional or pulmonary) administration. These amounts may be given in a single dose per day or in a number (such as two, three, four, five, or more) of sub-doses per day such that the total daily dose is the same. Similar dosages should be appropriate for treatment (including prophylaxis) of the other conditions referred herein for treatment. In general, determination of appropriate dosing can be readily arrived at by one skilled in medicine or the pharmacy art.

The present agent/composition can be administered to a mammal, preferably a human. Mammals include, but are not limited to, murines, rats, rabbit, simians, bovines, ovine, porcine, canines, feline, farm animals, sport animals, pets, equine, and primates.

The present disclosure also provides a method for inhibiting the growth of a cell in vitro, ex vivo or in vivo, where a cell, such as a cancer cell, is contacted with an effective amount of the present agent/composition as described herein.

Pathological cells or tissue such as hyperproliferative cells or tissue may be treated by contacting the cells or tissue with an effective amount of the present agent/composition. The cells, such as cancer cells, can be primary cancer cells or can be cultured cells available from tissue banks such as the American Type Culture Collection (ATCC). The pathological cells can be cells of a cancer as described herein, or a metastasis from a cancer as described herein (e.g., lung cancer, prostate cancer, breast cancer, hematopoietic cancer or ovarian cancer). The cells can be from a vertebrate, preferably a mammal, more preferably a human. U.S. Patent Publication No. 2004/0087651. Balassiano et al. (2002) Intern. J. Mol. Med. 10:785-788. Thorne, et al. (2004) Neuroscience 127:481-496. Fernandes, et al. (2005) Oncology Reports 13:943-947. Da Fonseca, et al. (2008) Surgical Neurology 70:259267. Da Fonseca, et al. (2008) Arch. Immunol. Ther. Exp. 56:267-276. Hashizume, et al. (2008) Neuroncology 10:112-120.

In vitro efficacy of the present composition can be determined using methods well known in the art. For example, the cytotoxicity of the present agent/composition may be studied by MTT [3-(4,5-dimethylthiazol-2-yl)-2,5-diphenyl tetrazolium bromide] cytotoxicity assay, MTT assay is based on the principle of uptake of MTT, a tetrazolium salt, by metabolically active cells where it is metabolized into a blue colored formazon product, which can be read spectrometrically. J. of Immunological Methods 65: 55 63, 1983. The cytotoxicity of the present agent/composition may be studied by colony formation assay. Functional assays for inhibition of VEGF secretion and IL-8 secretion may be performed via ELISA. Cell cycle block by the present agent/composition may be studied by standard propidium iodide (PI) staining and flow cytometry. Invasion inhibition may be studied by Boyden chambers. In this assay a layer of reconstituted basement membrane, Matrigel, is coated onto chemotaxis filters and acts as a barrier to the migration of cells in the Boyden chambers. Only cells with invasive capacity can cross the Matrigel barrier. Other assays include, but are not limited to cell viability assays, apoptosis assays, and morphological assays.

### EXAMPLES

The present disclosure can be better understood by reference to the following examples which are offered by way of illustration. The present disclosure is not limited to the examples given herein.

### Example 1

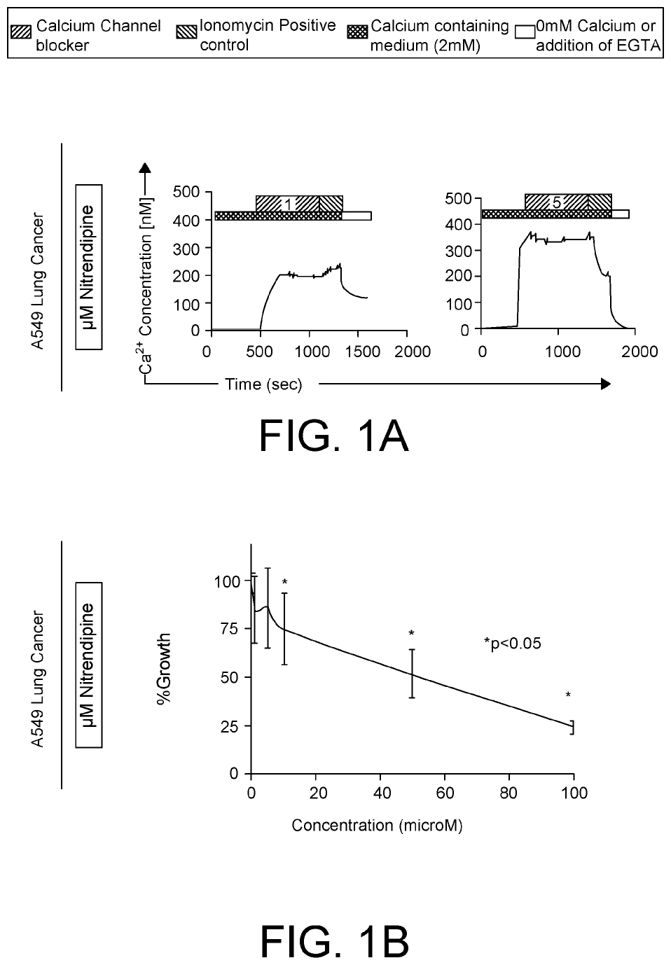

Example 1 was a measure of intracellular free calcium A549 lung cancer cells after treatment with nitrendipine as a function of time. The intracellular free calcium measurement was done using Fura-2-AM fluorescence measurement. Two concentrations of nitrendipine were studied, 1 μM and 5 μM. Ionomycin was used as the positive control for stimulation of calcium entry. Calcium was removed from the medium at the end of each experiment via addition of EGTA or replacement of the medium with calcium free medium. The calculation of the absolute calcium concentration was performed using a standard calcium curve. An average of 15 cells is shown. The results are illustrated in FIG. 1A. As illustrated, nitrendipine functions as an agonist for the entry of extracellular calcium into the A549 lung cancer cells. The cells maintained an increased concentration (relative to the absence of treatment with nitrendipine) of intracellular calcium for both 1 μM and 5 μM treatments of nitrendipine. The concentration of intracellular calcium increased with increasing nitrendipine concentration. At the 5 μM treatment, the concentration of intracellular calcium was greater than that of the positive control.

An XTT assay was performed 48 hours after the addition of nitrendipine at increasing concentrations. The cell proliferation was then calculated. Results are representative of at least three independent experiments. The results are illustrated in FIG. 1B. As illustrated, the percentage of cell growth decreases as the concentration of nitrendipine is increased. Without limitation by theory, it is theorized that nitrendipine lethally flooded the cells with calcium at the noted concentrations.

### Example 2

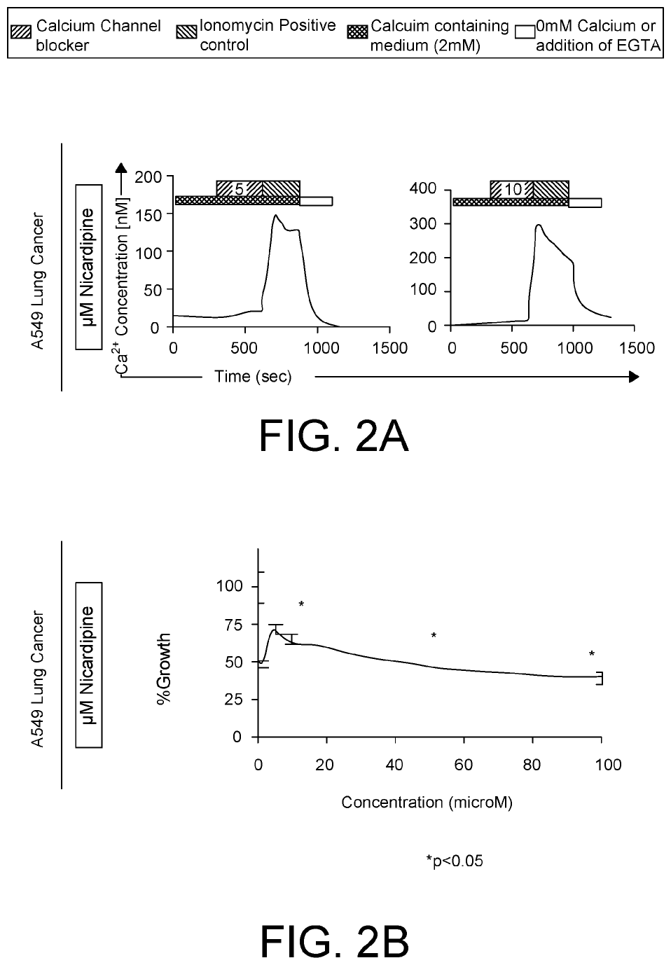

Example 2 was a measure of intracellular free calcium in A549 lung cancer cells after treatment with nicardipine as a function of time. The intracellular free calcium measurement was done using Fura-2-AM fluorescence measurement. Two concentrations of nicardipine were studied, 5 μM and 10 μM. Ionomycin was used as the positive control for stimulation of calcium entry. Calcium was removed from the medium at the end of each experiment via addition of EGTA or replacement of the medium with calcium free medium. The calculation of the absolute calcium concentration was performed using a standard calcium curve. An average of 15 cells is shown. The results are illustrated in FIG. 2A. As illustrated, nicardipine blocks the influx of extracellular calcium into the A549 lung cancer cells, and the cells maintained a low concentration of intracellular calcium for both 5 μM and 10 μM treatments of nicardipine.

An XTT assay was performed 48 hours after the addition of nicardipine at increasing concentrations. The cell proliferation was then calculated. Results are representative of at least three independent experiments. The results are illustrated in FIG. 2B. As illustrated, the percentage of cell growth decreases as the concentration of nicardipine is increased. Without limitation by theory, it is theorized that the nicardipine-initiated block of the entry of extracellular free calcium inhibits the cell's ability to undergo or successfully finish cytokinesis, and this may lead to cell death.

### Example 3

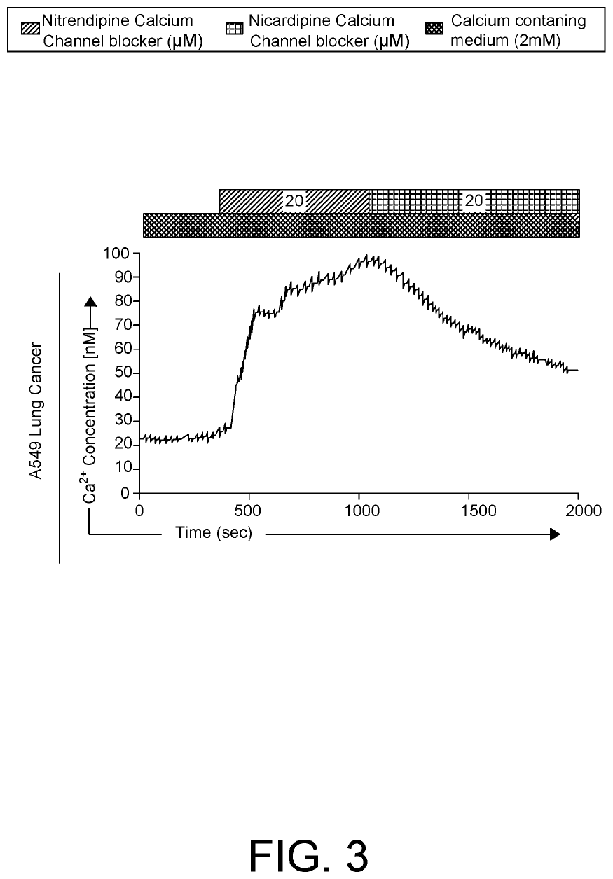

Example 3 was a measure of intracellular free calcium in A549 lung cancer cells after consecutive treatments with nitrendipine and nicardipine as a function of time. The intracellular free calcium measurement was done using Fura-2-AM fluorescence measurement. The individual concentrations of nitrendipine and nicardipine were 20 μM. The calculation of the absolute calcium concentration was performed using a standard calcium curve. An average of 15 cells is shown. The results are illustrated in FIG. 3. As illustrated, nitrendipine floods the cells with calcium as described above; however, the administration of nicardipine resulted in a block of the influx of extracellular calcium triggered by nitrendipine and the resulting intracellular calcium concentration decreased after nicardipine treatment as illustrated.

### Example 4

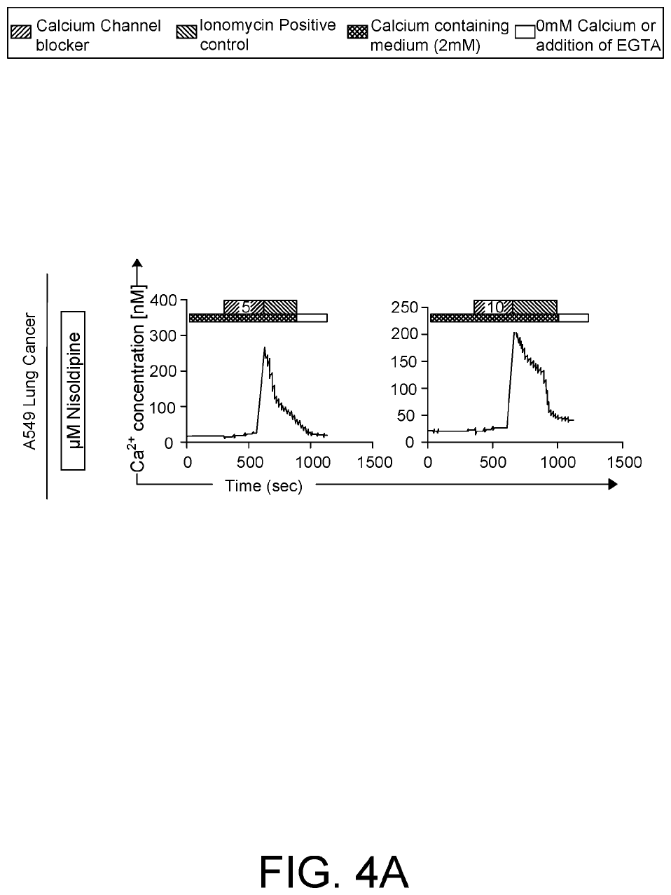

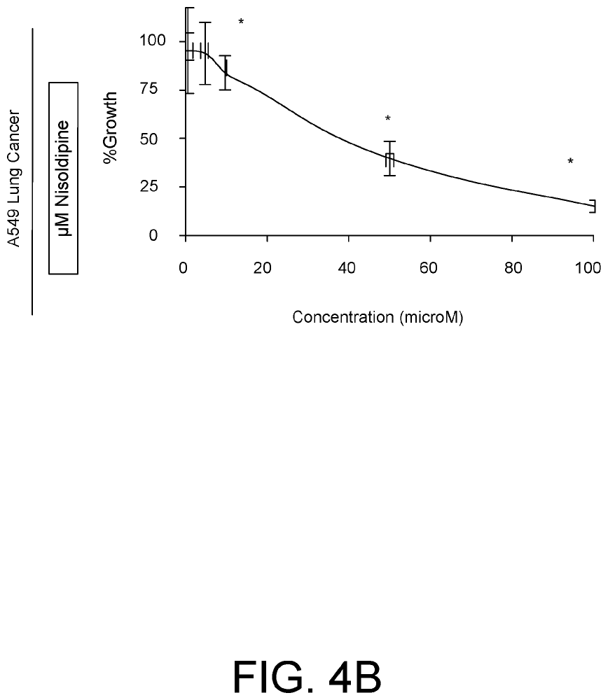

Example 4 was a measure of intracellular free calcium in A549 lung cancer cells after treatment with nisoldipine as a function of time. The intracellular free calcium measurement was done using Fura-2-AM fluorescence measurement. Two concentrations of nisoldipine were studied, 5 μM and 10 μM. Ionomycin was used as the positive control for stimulation of calcium entry. Calcium was removed from the medium at the end of each experiment via addition of EGTA or replacement of the medium with calcium free medium. The calculation of the absolute calcium concentration was performed using a standard calcium curve. An average of 15 cells is shown. The results are illustrated in FIG. 4A. As illustrated, nisoldipine blocks the influx of extracellular calcium into the A549 lung cancer cells, and the cells maintained, a low concentration of intracellular calcium for both 5 μM and 10 μM treatments of nisoldipine.

An XTT assay was performed 48 hours after the addition of nisoldipine at increasing concentrations. The cell proliferation was then calculated. Results are representative of at least three independent experiments. The results are illustrated in FIG. 4B. As illustrated, the percentage of cell growth decreases as the concentration of nisoldipine is increased. Without limitation by theory, it is theorized that the nisoldipine-initiated block of the entry of extracellular free calcium inhibits the cell's ability to undergo or successfully finish cytokinesis and this may lead to cell death.

### Example 5

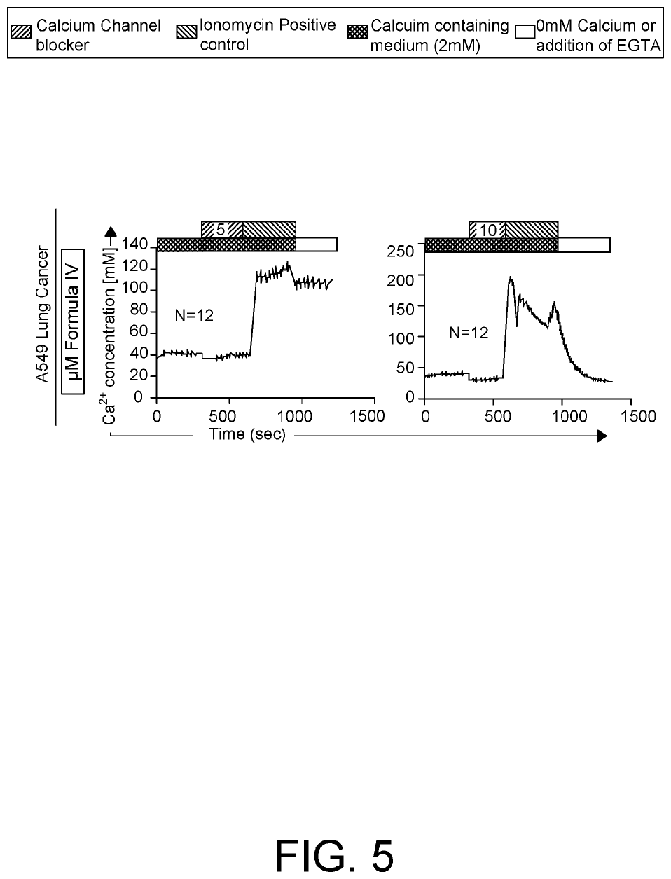

Example 5 was a measure of intracellular free calcium in A549 lung cancer cells after treatment with the drug of Formula IV as a function of time. The intracellular free calcium measurement was done using Fura-2-AM fluorescence measurement. Two concentrations of the drug of Formula IV were studied, 5 μM and 50 μM. Ionomycin was used as the positive control for stimulation of calcium entry. Calcium was removed from the medium at the end of each experiment via addition of EGTA or replacement of the medium with calcium free medium. The calculation of the absolute calcium concentration was performed using a standard calcium curve. An average of 15 cells is shown. The results are illustrated in FIG. 5. As illustrated, the drug of Formula IV blocks the influx of extracellular calcium into the A549 lung cancer cells, and the cells maintained a low concentration of intracellular calcium for both 5 μM and 50 μM treatments. As compared with nitrendipine in Example 1, it appears that the substitution of a phenyl for a nitrophenyl at the 4 position of the dihydropyridine results in a loss of the ability o flood the A549 lung cancer cells with calcium. These results provide a strong indication that the mechanism for the large increase in intracellular calcium from nitrendipine treatment may be due to the presence of the nitro functional group at the 3 position on the phenyl group.

### Example 6

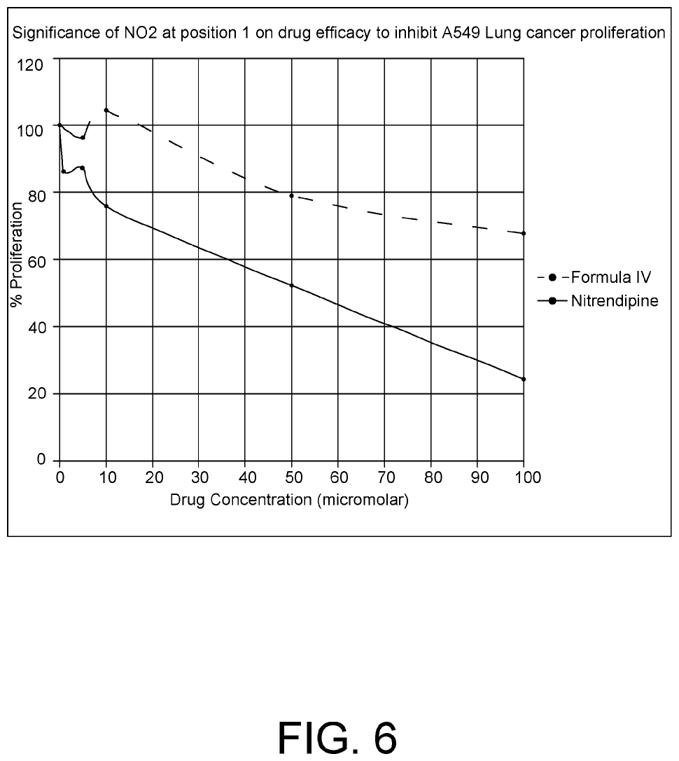

Example 6 was a study to determine the effect of treatment with nitrendipine and the dihydropyridine of Formula IV above on the proliferation of cancer cells. NSCLC A549 lung cancer cells were incubated with various concentrations of nitrendipine and the dihydropyridine of Formula IV ranging from 1 μM to 100 μM. 5000 cells were plated in RPMI medium per well and incubated with either the individual drugs for 24 hours. An XTT assay was then performed. The cell proliferation was then calculated. The results of one of three independent experiments is presented in Table 1 below. FIG. 6 is a graphical representation of the results.

TABLE 1
The nitrendipine and the drug of Formula
IV on lung cancer cell proliferation

| | μM | | | |
|---|---|---|---|---|
| | Concen- | Avg. % | | |
| | tration | proliferation | Std. dev | T-test |
| | 0 | 100 | 15.82803109 | |
| Formula IV | 5 | 96.39532108 | 12.73511079 | 0.27741439 |
| | 10 | 104.2253521 | 17.83055777 | 0.256868817 |
| | 50 | 79.0642158 | 12.88974267 | 0.000683911 |
| | 100 | 67.79661017 | 6.276123839 | 1.00649E−06 |
| Nitrendipine | 0 | 100 | 5 | |
| | 1 | 86.23753117 | 17.58059732 | 0.115005532 |
| | 5 | 87.1882793 | 20.7406768 | 0.149290861 |
| | 10 | 75.96633416 | 18.45672356 | 0.025854961 |
| | 50 | 52.21321696 | 12.68738801 | 0.00016844 |
| | 100 | 24.42331671 | 3.307304774 | 4.45086E−07 |

As observed, the drugs were effective in inhibiting cell proliferation of the lung cancer cell samples.

### Example 7

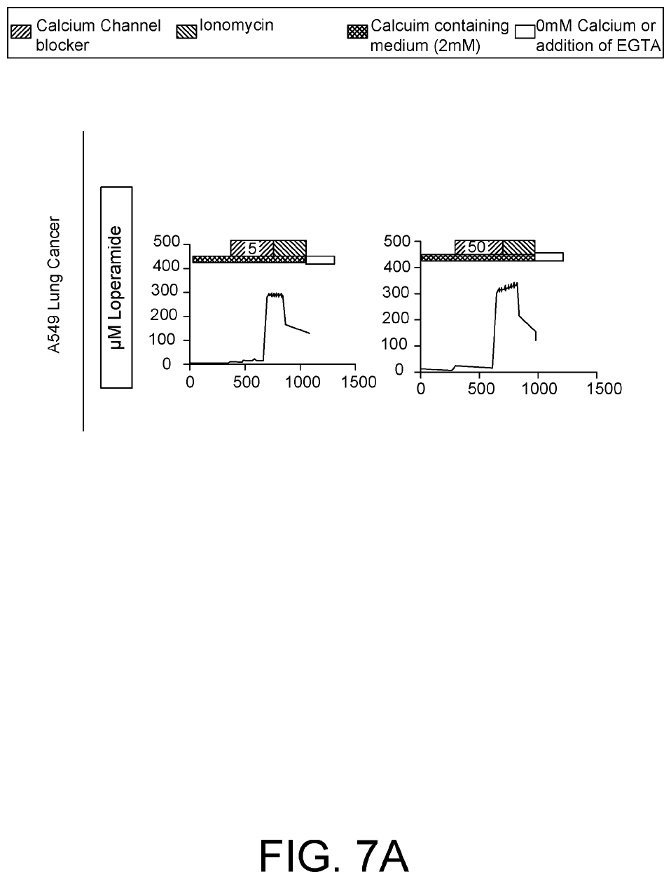

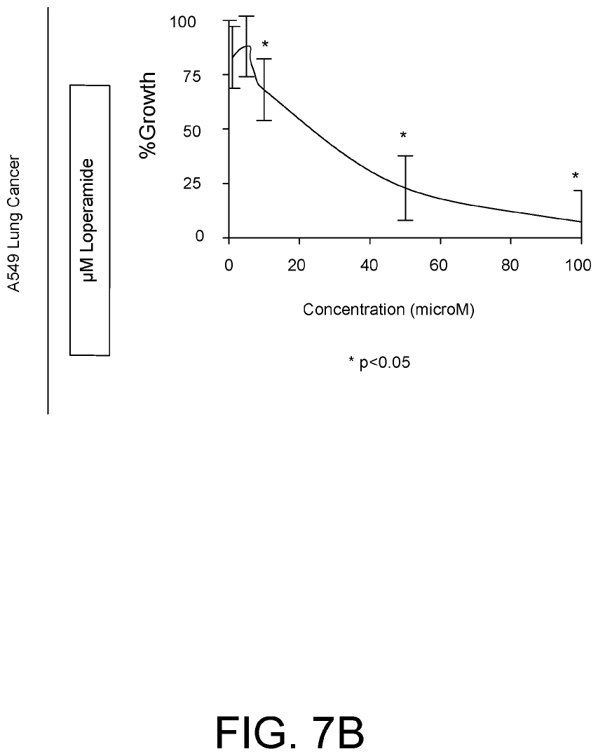

Example 7 was a measure of intracellular free calcium in A549 lung cancer cells after treatment with loperamide as a function of time. The intracellular free calcium measurement was done using Fura-2-AM fluorescence measurement. Two concentrations of loperamide were studied, 5 μM and 50 μM. Ionomycin was used as the positive control for stimulation of calcium entry. Calcium was removed from the medium at the end of each experiment via addition of EGTA or replacement of the medium with calcium free medium. The calculation of the absolute calcium concentration was performed using a standard calcium curve. An average of 15 cells is shown. The results are illustrated in FIG. 7A. As illustrated, loperamide blocks the influx of extracellular calcium into the A549 lung cancer cells, and the cells maintained a low concentration of intracellular calcium for both 5 μM and 50 μM treatments of loperamide.

An XTT assay was performed 48 hours after the addition of loperamide at increasing concentrations. The cell proliferation was then calculated. Results are representative of at least three independent experiments. The results are illustrated in FIG. 7B. As illustrated, the percentage of cell growth decreases as the concentration of loperamide is increased. Without limitation by theory, it is theorized that the loperamide-initiated block of the entry of extracellular free calcium inhibits the cell's ability to undergo or successfully finish cytokinesis, and this may lead to cell death.

### Example 8

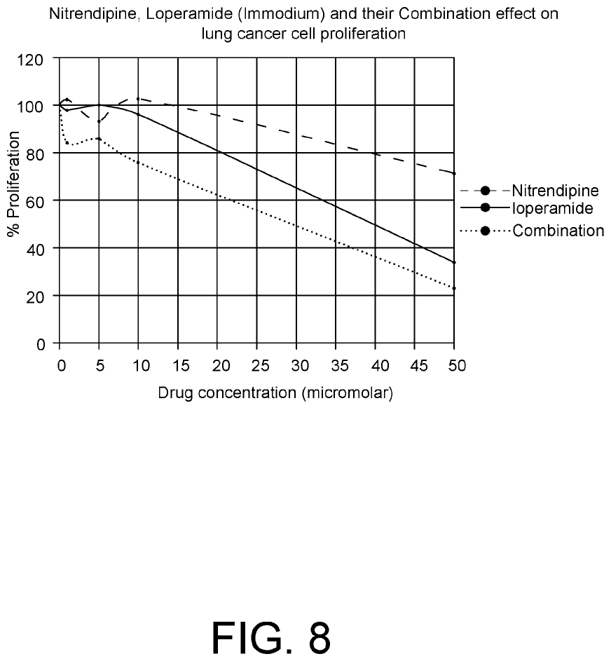

Example 8 was a study to determine the effect of the combination of calcium blockers on the proliferation of cancer cells. NSCLC A549 lung cancer cells were incubated with various concentrations of a combination of calcium blockers ranging from 1 μM to 50 μM. In this specific example, the combination of loperamide and the dihydropyridine, nitrendipine, was used. Specifically, 5000 cells were plated in RPMI medium per well and incubated with either the individual compound or the combination of loperamide and the dihydropyridine for 24 hours. An XTT assay was then performed. The cell proliferation was then calculated. Results are representative of at least three independent experiments. The raw data is presented in Table 2 below. FIG. 8 is a graphical representation of the results.

TABLE 2
The combination effect of loperamide and nitrendipine
on lung cancer cell proliferation

| | μM | | | |
|---|---|---|---|---|
| | Concen- | Avg. % | | |
| | tration | proliferation | Std. dev | T-test |
| | 0 | 100 | 8.58087308 | |
| Nitrendipine | 1 | 102.33 | 15.64 | 0.35878714 |
| | 5 | 93.35 | 9.75 | 0.08479743 |
| | 10 | 102.58 | 15.57 | 0.34398005 |
| | 50 | 71.43 | 11.13 | 2.5097E−05 |
| Loperamide | 1 | 98.07 | 16.11 | 0.38448766 |
| | 5 | 99.77 | 17.03 | 0.48671762 |
| | 10 | 96.09 | 14.78 | 0.26413712 |
| | 50 | 34.04 | 4.15 | 7.194E−12 |
| Combination | 1 | 84.15 | 11.16 | 0.04926654 |
| | 5 | 85.77 | 13.13 | 0.00437743 |
| | 10 | 75.88 | 18.25 | 0.000298 |
| | 50 | 23.16 | 5.93 | 1.4427E−14 |

As observed, the combination of loperamide and nitrendipine has a greater than additive effect on the cell proliferation of the lung cancer cell samples. This synergistic effect is especially pronounced at the lower concentrations of the combination.

### Example 9

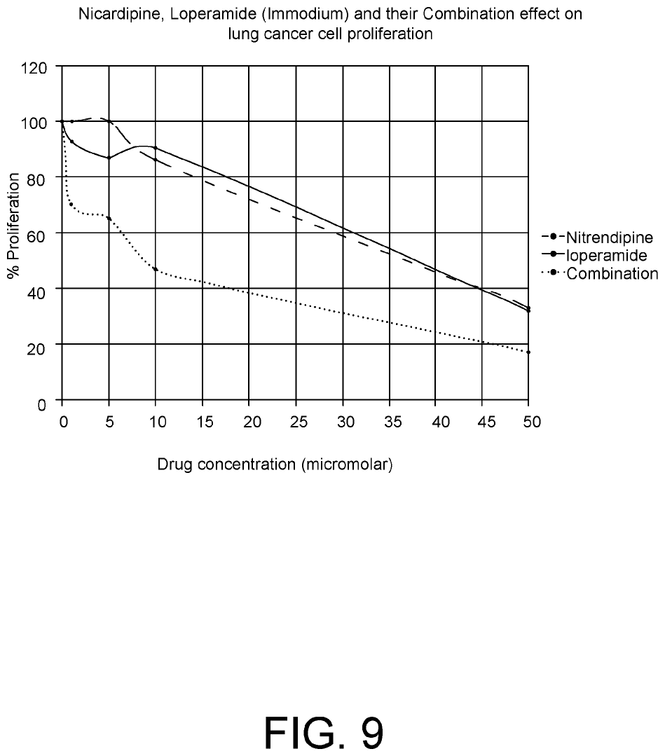

Example 9 is a study to determine the effect of the combination of calcium blockers on the proliferation of cancer cells. NSCLC A549 lung cancer cells were incubated with various concentrations of a combination of calcium blockers ranging from 1 μM to 50 μM. In this specific example, the combination of loperamide and the dihydropyridine, nicardipine, was used. Specifically, 5000 cells were plated in RPMI medium per well and incubated with either the individual compound or the combination of loperamide and the dihydropyridine for 24 hours. An XTT assay was then performed. The cell proliferation was then calculated. Results are representative of at least three independent experiments. The raw data is presented in Table 3 below. FIG. 9 is a graphical representation of the results.

TABLE 3
The combination effect of loperamide and nicardipine
on lung cancer cell proliferation

| | μM | | | |
|---|---|---|---|---|
| | Concen- | Avg. % | | |
| | tration | proliferation | Std. dev | T-test |
| | 0 | 100 | 8.58087308 | |
| Nicardipine | 1 | 99.90 | 15.78 | 0.48727697 |
| | 5 | 100.23 | 19.66 | 0.08371004 |
| | 10 | 86.20 | 26.50 | 1.8273E−15 |
| | 50 | 32.97 | 3.49 | 0.00456208 |
| Loperamide | 1 | 92.73 | 21.08 | 0.17691732 |
| | 5 | 86.97 | 24.14 | 0.07708557 |
| | 10 | 90.33 | 13.24 | 0.03394176 |
| | 50 | 32.03 | 3.67 | 2.0389E−15 |
| Combination | 1 | 70.01 | 13.95 | 2.7192E−06 |
| | 5 | 65.10 | 26.98 | 6.3805E−05 |
| | 10 | 46.84 | 23.73 | 8.2184E−09 |
| | 50 | 16.99 | 4.40 | 4.1121E−15 |

### Example 10

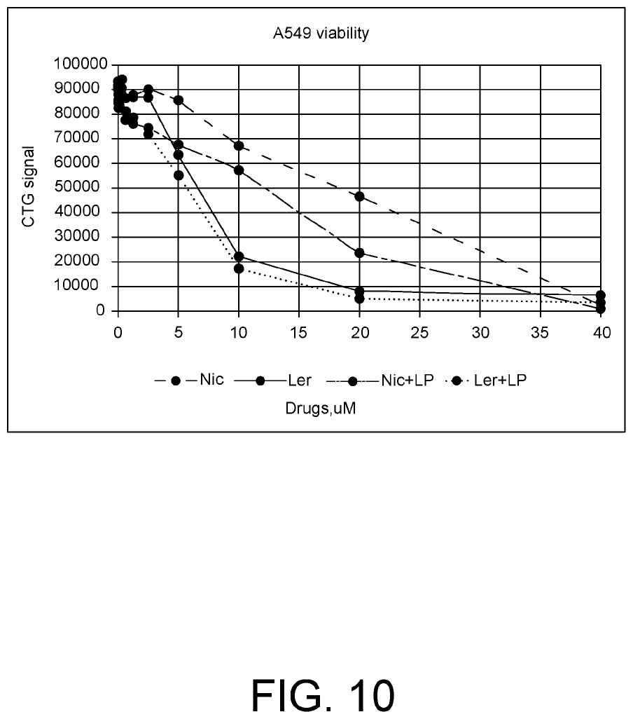

Example 10 (see also FIG. 10) is a study to determine the effect of the combination of calcium blockers on the proliferation of cancer cells. NSCLC A549 lung cancer cells were incubated with various concentrations of a combination of calcium blockers ranging from 1 μM to 50 μM. In this specific example, the combination of loperamide and the dihydropyridine, azelnidipine, niguldipine, lercanidipine or nicardipine were used. Specifically, 5000 cells were plated in RPMI medium per well and incubated with either the individual compound or the combination of loperamide and the dihydropyridine for 72 hours. An XTT assay was then performed. The cell proliferation was then calculated. Results are representative of at least three independent experiments. IC50 was calculated from the % inhibition data.

TABLE 4
Combination effects of loperamide and dihydropyridine
on lung cancer cell proliferation

| | Plus DMSO (Control), | Plus Loperamide, |
|---|---|---|
| Compound | IC-50 | IC-50 |
| Loperamide | 12.2 | |
| Azelnidipine | 8.5 | 4.8 |
| Niguldipine | 5.37 | 3.12 |
| Manidipine | 8.2 | |
| Lercanidipine | 7.7 | 3.95 |
| Efonidipine | 10.2 | |
| Nicardipine | 13.7 | 6.7 |
| Benidipine | 18 | |
| Clinidipine | 41 | |
| Nifedipine | No Activity | |
| Amlodipine | 22 | |
| Nimodipine | No Activity | |
| Isradipine | 26 | |
| Felodipine | 26 | |
| Clevidipine | 23 | |
| Lacidipine | 16 | |
| Nilvadipine | 17.6 | |
| Verapamil | No Activity | |
| Diltiazem | No Activity | |

One or more illustrative examples incorporating the examples disclosed herein are presented. Not all features of a physical implementation are described or shown in this application for the sake of clarity. Therefore, the disclosed, systems and methods are well adapted to attain the ends and advantages mentioned, as well as those that are inherent therein. The particular examples disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown other than as described in the claims below. It is therefore evident that the particular illustrative examples disclosed above may be altered, combined, or modified, and all such variations are considered within the scope of the present disclosure. The systems and methods illustratively disclosed herein may suitably be practiced in the absence of any element that is not specifically disclosed herein and/or any optional element disclosed herein.

Although the present disclosure and its advantages have been described in detail, it should be understood that various changes, substitutions and alterations can be made herein without departing from the spirit and scope of the disclosure as defined by the following claims.

1. A method of treating cancer or inhibiting growth of cancer cells in a subject, comprising administering an effective amount of at least one dihydropyridine to the subject.

2. (canceled)

3. A method of treating cancer or inhibiting growth of cancer cells in a subject, wherein the subject is afflicted with a cancer expressing a Cav1.3 calcium channel, the method comprising administering an effective amount of at least one dihydropyridine to the subject.

4. (canceled)

5. The method of claim 1, wherein the dihydropyridine is selected from the group consisting of lercanidipine, manidipine, nitrendipine, nicardipine, nisoldipine, and any combination thereof.

6. The method of claim 1, wherein the cancer is a carcinoma.

7. The method of claim 1, wherein the cancer is a lung cancer.

8. The method of claim 7, wherein the cancer is a non-small cell lung cancer.

9. The method of claim 1, wherein the cancer is a tumor and wherein the effective amount is sufficient to reduce the size or mass of the tumor.

10. The method of claim 1, further comprising identifying the cancer in the subject.

11. (canceled)

12. The method of claim 10, wherein the identifying the cancer in the subject comprises identifying whether the cancer expresses an isoform of a Cav1.3 channel.

13. The method of claim 1, wherein the dihydropyridine is administered orally.

14. The method of claim 1, wherein the effective amount ranges from about 0.1 mg/day to about 100 mg/day.

15. The method of claim 1, wherein the dihydropyridine is administered topically, intratumorally, or through a pulmonary administration.

16. The method of claim 15, wherein the effective amount ranges from about 5 μM to about 50 μM.

17. (canceled)

18. (canceled)

19. (canceled)

20. (canceled)

21. A pharmaceutical composition for the treatment of cancer, the pharmaceutical composition comprising an effective amount of at least one dihydropyridine.

22. The pharmaceutical composition of claim 21, wherein the dihydropyridine is selected from the group consisting of lercanidipine, manidipine, nitrendipine, nicardipine, nisoldipine, and any combination thereof.

23. The pharmaceutical composition of claim 21, wherein the pharmaceutical composition is configured to be administered orally.

24. The pharmaceutical composition of claim 23, wherein the effective amount ranges from about 0.1 to about 100 mg.

25. The pharmaceutical composition of claim 21, wherein the pharmaceutical composition is configured to be administered topically, intratumorally, or through a pulmonary administration.

26. The pharmaceutical composition of claim 25, wherein the dihydropyridine has a concentration ranging from about 5 μM to about 50 μM.

27. The pharmaceutical composition of claim 21, wherein the dihydropyridine is nitrendipine, wherein the pharmaceutical composition is configured to be administered locally, and wherein the dihydropyridine has a concentration ranging from about 5 μM to about 50 μM.
